(19) United States
(12) Patent Application Publication
(10) Pub. No.: US 2023/0044268 A1
(43) Pub. Date: Feb. 9, 2023

(54) AN IMPROVED PROCESS FOR PREPARATION OF LIRAGLUTIDE

(71) Applicant: SHILPA MEDICARE LIMITED, RAICHUR (IN)

(72) Inventors: KISHOR VALLABH PATIL, Raichur (IN); CHANDRASEN BABASAHEB CHANDWAD, Raichur (IN)

(73) Assignee: SHILPA MEDICARE LTD, RAICHUR (IN)

(21) Appl. No.: 17/784,126

(22) PCT Filed: Dec. 21, 2020
(86) PCT No.: PCT/IB2020/062265

§ 371 Date: Jun. 10, 2022

(30) Foreign Application Priority Data
Dec. 23, 2019 (IN) 201941053392

(51) Int. Cl. C07K 14/605

(52) U.S. Cl. CPC C07K 14/605

(57) ABSTRACT

The present invention relates to an improved process for the preparation of Liraglutide. The present invention further related an improved process for the preparation of substantially pure material having a purity of greater than or equal to 99.5% by HPLC.

### FIELD OF THE INVENTION

The present invention relates to an improved process for the preparation Liraglutide or its pharmaceutically acceptable salts.

### BACKGROUND OF THE INVENTION

Liraglutide is marketed under the brand name VICTOZA® in the U.S, India, Canada, Europe and Japan. The peptide precursor of liraglutide, produced by a process that includes expression of recombinant DNA in Saccharomyces cerevisiae, has been engineered to be 97% homologous to native human GLP-1 by substituting arginine for lysine at position 34. Liraglutide is made by attaching a C-16 fatty acid (palmitic acid) with a glutamic acid spacer on the remaining lysine residue at position 26 of the peptide precursor. The molecular formula of liraglutide is C₁₇₂H₂₆₅N₄₃O₅₁ and the molecular weight is 3751.2 Daltons. The structural formula (FIG. 1) is:

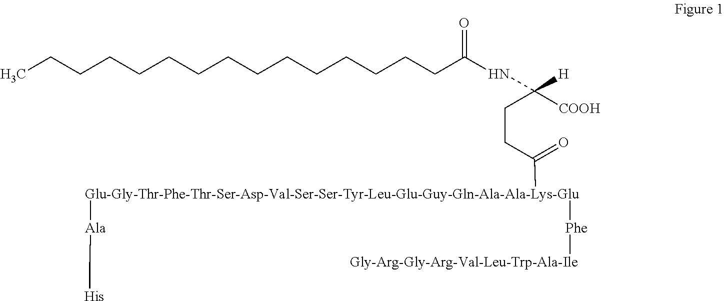

As per the scientific discussion “The liraglutide drug substance manufacturing process has adequately been described and a flow chart has been provided. Briefly, it consists of the following main steps: fermentation of yeast cells, recovery and purification of liraglutide precursor, acylation of the precursor and further purification of liraglutide to drug substance.”

Liraglutide is first disclosed in U.S. Pat. Nos. 6,268,343B1 and 6,458,924B2, in which Liraglutide preparation by biological route, it mainly through genetic engineering and other biological methods of preparation. Liraglutide preparation by biological route which involves technical difficulties and having limitation in attachment of Palmitoyl-Glu-spacer. The process generates biological impurities including unwanted proteins, cell debris, host DNA and genetic material which required immense purification process, therefore production costs increases. The disadvantage of the process described in U.S. Pat. No. 6,268,343 is that the N-terminal of GLP-1(7-37)-OH is not protected, which leads to generation of impurities. Additional purification steps are required to remove these impurities, and it makes Liraglutide high cost and not suitable for large scale production.

In U.S. Pat. Nos. 6,268,343B1 and 6,458,924B2 the purification is performed using reverse-phase HPLC for intermediate GLP-1(7-37)-OH, followed by reaction with Nα-alkanoyl-Glu(ONSu)-OtBu in liquid phase. In such process, the N-terminal of GLP-1(7-37)-OH is not protected and protective groups for the side chains are all removed, leading to formation of a great amount of impurities, difficulties in purification and complicated operation steps. The prior art process for the preparation of the liraglutide involves purification steps, long synthesis cycle, large amount of waste liquid which is not environmentally friendly, and involves high amount of solvent like acetonitrile, which is cumbersome in large-scale production.

U.S. Pat. No. 7,572,884 disclosed a process for preparing Liraglutide by recombinant technology followed by acylation and removal of N-terminal extension.

U.S. Pat. No. 9,260,474 discloses process for the preparation of solid phase synthesis of Liraglutide comprises lysine
a) the presence of the activator system, solid phase carrier and by resin Fmoc protection N end obtained by coupling of glycine (Fmoc-Gly-OH) Fmoc-Gly-resin;
b) by solid phase synthesis, prepared in accordance with the sequentially advantage Liraglutide principal chain N end of the coupling with Fmoc protected amino acid side chain protection and, wherein the lysine using Fmoc-Lys (Alloc)-OH;
c) Alloc getting rid of the lysine side chain protecting group;
d) by solid phase synthesis, the lysine side chain coupling Palmitoyl-Glu-OtBu;
e) cracking, get rid of protecting group and resin to obtain crude Liraglutide;
f) purification, freeze-dried, to obtain Liraglutide.

Even through, the above-mentioned prior art discloses diverse processes for the preparation of Liraglutide, they are often not amenable on commercial scale because of expensive amino acid derivatives such as pseudo prolines used in those processes.

International publications WO 2019170918 and WO 2019170895 disclosed a process for the preparation of liraglutide comprising enzymatically coupling of a peptide C-terminal ester or thioester comprising a first peptide fragment with a peptide nudeophile having an N-terminally unprotected amine comprising a second peptide fragment, wherein enzyme coupling is catalyzed by ligase.

As the process described in this patent application involves the use of enzyme ligase, which additionally requires a purification of enzyme step and is difficult to handle the content of enzyme in the final liraglutide.

WO 2016/046753 discloses methods for synthesizing GLP-1 peptides, including Liraglutide and Semaglutide, which comprise a final coupling step in which at least two fragments are coupled at a terminal Gly residue, wherein at least one of the fragments is prepared by the coupling of at least two sub-fragments. By way of example, WO 2016/046753 discloses coupling fragment (1-4) and fragment (5-31) in solid state or in solution. Fragment (5-31) can be prepared by coupling fragment (5-16) with fragment (17-31). Fragment (5-16) itself can be prepared by coupling fragment (5-12) with fragment (13-16). Coupling with a terminal Gly, for example, at Gly4 or Glyl6, avoids racemization.

WO 2019153827 disclosed a process for the preparation of a liraglutide intermediate polypeptide GLP-1(7-37), comprising constructing a recombinant liraglutide engineered bacteria, expressing a liraglutide intermediate fusion protein in the form of an inclusion body by means of E. coli induction. The prepared liraglutide intermediate polypeptide has a purity reaching 87% or higher and a yield greater than 85%.

U.S. Pat. No. 10,344,069 disclosed a process for the preparation of Liraglutide, comprises synthesis of suitable fragments (protected) by solid phase peptide synthesis; followed by coupling of the suitable fragments on solid support; concurrently cleaving the protected peptide from the solid support and de-protecting the peptide; followed by purification of Liraglutide (crude) on reverse phase HPLC and isolating pure Liraglutide.

Further, WO 2016/005960 and WO 2019069274 disclosed a process for the preparation of a liraglutide comprised sequential development of fragments followed by coupling.

The main disadvantage of these fragment coupling, fragment condensation into each of solid phase segment needed excess fragments to condensation, it resulted in the serious waste of peptide fragments hence it resulting into high cost of synthesis. Solid phase segment condensation needed synthesis in multiple reactions and it condensation having limitation in resin substitution, and it generate large amount of waste. Also the solid phase fragment condensation method of synthesis generates impurities of fragments during the condensation due to unreacted fragments and it also leads to formation of optically impure Liraglutide and it is difficult to purify to its homogeneity

In Patent WO2013/037266 describes solid phase synthesis of Liraglutide synthesis by using Alloc protected Lysine in linear sequence. After completion of liraglutide peptide sequence attachment of peptide linker is must to complete the molecule. The patent described the uses tetrakis (triphenylphosphine) palladium to remove Alloc and then attachment of Peptide linker. This process is costlier because use of tetrakis (triphenylphosphine) palladium to deprotect lysine.

In Patent WO2014/199397 describes the use of Dde protected Lysine substrate and it's selectively deprotection by using hydrazine. As hydrazine can also remove Fmoc groups as well as Dde groups. The basic nature of hydrazine removes Fmoc protections as lead to form unexpected side impurities during synthesis if traces of hydrazine in synthesis.

In Patent WO2015/100876 describe resin solid phase carrier is 2-CTC resin and activated system selected from the DIEA, TMP or NMM for CTC resin and DIC, HOBt and DMAP for king resin, and the Fmoc-Gly resin is 0.10-0.35 mmol./g Substitution degree of Fmoc-Gly on both resins, the lower substitution of Fmoc Gly on resin which results in low yield.

Further in the same patent disclosed the molar ratio as 1:3:3:3:3 and 1:5:5:5:5, it clear indicates that amino acids are is used in large excess which results into higher production cost.

Hence, there remains a need to provide simple, cost effective, scalable and robust processes for the preparation of Liraglutide involving commercially viable amino acid derivatives and reagents.

In view of the above it is pertinent to note that there is a need to develop new process for the preparation of Liraglutide having further improved physical and/or chemical properties besides high purity levels. Hence it was thought worthwhile by the inventors of the present application to explore novel process for the preparation of Liraglutide, which may further improve the characteristics of drug Liraglutide and in developing the substantially pure Liraglutide.

Exploring new process for developing a stable and pure form of Liraglutide, which are amenable to scale up for pharmaceutically active useful compounds in the preparation of Liraglutide may thus provide an opportunity to improve the drug performance characteristics of products such as purity and solubility. Hence, inventors of the present application report a process for the preparation of a stable and substantially pure form of Liraglutide, which may be industrially amenable and usable for preparing the corresponding pharmaceutical compositions.

The present invention provides an improved process for the preparation of substantially pure Liraglutide, wherein substantially pure material having a purity of greater than or equal to 99.5% by HPLC and meeting the quality of ICH guidelines. Liraglutide obtained by the process of the present invention is chemically stable and has good dissolution properties.

In view of the above and to overcome the prior-art problems the present inventors had now developed an improved process for the preparation of substantially pure Liraglutide, using industrially feasible and viable process, with the use of industrially friendly solvents, which does not include tedious work up and time lagging steps.

### OBJECTIVE OF THE INVENTION

The main objective of the invention relates to a process for the preparation of Liraglutide.

Yet another objective of the invention relates to an improved process for the preparation of substantially pure material having a purity of greater than or equal to 99.5% by HPLC.

Yet another objective of the invention relates to an improved process for the preparation of substantially pure material having a purity of greater than or equal to 99.5% by HPLC free of process related impurities.

### SUMMARY OF THE INVENTION

The main objective of the invention relates to an improved process for solid phase synthesis of highly pure Liraglutide, comprises:
a) reacting protected glycine with resin in the presence of an activating agent and a solvent to form Fmoc-Gly-resin and followed by capping with acetic anhydride in pyridine;
b) deprotection of Fmoc-Gly-resin, followed by solid-phase synthesis, amino acids with N-terminal Fmoc protection and side chain protection are sequentially coupled based on the sequence of peptide backbone of liraglutide, wherein purified Fmoc-Lys(Pal-Glu(OtBu)-OH is employed in place of Lysine; wherein the amino acids coupling is performed at 25-30° C. and involves 2 molar equivalent except amino acid coupling in position at 15, 16, 18, 22, 24, 25 28, and 31 coupling involves 2 to 3 molar equivalent and performed at 35-60° C. in presence of activating agent and solvent; and
c) Liraglutide is finally obtained by purification and lyophilizing; wherein the purification is performed by a reverse-phase high performance liquid chromatography using a reverse-phase C8 or C18 column using ammonium salts and ammonium buffers in first purification and followed by 0.1% TFA in water, acetonitrile or mixture thereof.

Another objective of the invention relates to an improved process for solid phase synthesis of highly pure Liraglutide, comprises treating crude liraglutide is finally obtained by purification and lyophilizing; wherein the purification is performed by a reverse-phase high performance liquid chromatography using a reverse-phase C8 or C18 column using ammonium buffer and ammonium salts and 0.1% TFA in water, acetonitrile or mixture thereof.

### DETAILED DESCRIPTION OF THE INVENTION

The present invention relates to an improved process for solid phase synthesis of highly pure Liraglutide, comprises reacting protected glycine with resin; in the presence of an activating agent and a solvent selected from the group consisting of DMF, DCM, NMP, Acetonitrile, TFA, Piperdine, Pyridine, Diethyl Ether, Diisopropyl Ether, Methyl tertiary Butyl Ether, Ethyl acetate, Dimethyl sulphoxide, Diisopropyl ethylamine, hexane, water and combination thereof; to form Fmoc-Gly-resin and followed by capping with acetic anhydride in pyridine.

The resin used in the present invention involves the use of Wang resin, which is employed as the resin solid phase support, and said Fmoc-Gly-resin with substitution degree in the range from 0.5 to 0.75 mmol/g.

The activating agent used in the present invention selected from DIC (Diisopropylcarbodiimide), DCC (N,N′-Dicyclohexylcarbodiimide), Ethylcyano (hydroxyimino)acetate, DIPEA (Diisopropyl ethylamine), HCTU (O-(1H-6-Chlorobenzotriazole-1-yl)-1,1,3,3-tetramethyluronium hexafluorophosphate), HATU (1-[Bis(dimethylamino)methylene]-1H-1,2,3-triazolo[4,5-b]pyridinium3-oxide hexafluorophosphate.

Capping of unreacted free amines involves stirring the amino acid in presence of two equivalent of acetic anhydride, two equivalents of pyridine in DMF for 30 minutes to 60 minutes, followed by washing with DMF.

The obtained capped Fmoc-Gly-resin undergo deprotection in presence of 20% piperidine in DMF followed by solid-phase synthesis, amino acids with N-terminal Fmoc protection and side chain protection are sequentially coupled based on the sequence of peptide backbone of liraglutide, wherein purified Fmoc-Lys(Pal-Glu(OtBu)-OH is employed in place of Lysine; wherein the amino acids coupling is performed at a temperature ranging from 25-30° C. and involves 2 molar equivalent except amino acid coupling in position at 15, 16, 18, 22, 24, 25, 28 and 31 coupling involves 2 to 3 molar equivalent and performed at a temperature ranging from 35-60° C. in presence of activating agent selected from DIC (Diisopropylcarbodiimide), DCC (N,N′-Dicyclohexylcarbodiimide) Ethylcyano (hydroxyimino) acetate, DIPEA (Diisopropyl ethylamine), HCTU (O-(1H-6-Chlorobenzo triazole-1-yl)-1,1,3,3-tetramethyl uronium hexafluoro phosphate), HATU (1-[Bis(dimethylamino)methylene]-1H-1,2,3-triazolo[4,5-b]pyridinium3-oxide hexafluoro phosphate; and solvent selected from DMF, DCM, NMP, Acetonitrile, TFA, Piperdine, Pyridine, Diethyl Ether, Diisopropyl Ether, Methyl tertiary Butyl Ether, Ethyl acetate, Dimethyl sulphoxide, Diisopropyl ethylamine, hexane, water and combination thereof; finally crude liraglutide is obtained by cleavage, and removal of the protective groups and the resin

The sub-sequential coupling involves the use of amino acids Fmoc-Arg(Pbf)-OH (Arg), Fmoc-Gly-OH (Gly), Fmoc-Arg(Pbf)-OH (Arg), Fmoc-Val-OH (Val), Fmoc-Leu-OH (Leu), Fmoc-Trp(Boc)-OH (Trp), Fmoc-Ala-OH (Ala), Fmoc-Ile-OH (Ile), Fmoc-Phe-OH (Phe), Fmoc-Glu(OtBu)-OH (Glu), Fmoc-Lys(Pal-Glu(OtBu))-OH, Fmoc-Ala-OH (Ala), Fmoc-Ala-OH (Ala), Fmoc-Gln(Trt)-OH (Gln), Fmoc-Gly-OH, Fmoc-Glu (OtBu)-OH, Fmoc-Leu-OH, Fmoc-Tyr (tBu)-OH, Fmoc-Ser(tBu)-OH, Fmoc-Ser(tBu)-OH (Ser), Fmoc-Val-OH, Fmoc-Asp (OtBu)-OH (Asp), Fmoc-Ser (tBu)-OH, Fmoc-Thr (tBu)-OH, Fmoc-Phe-OH, Fmoc-Thr (tBu)-OH, Fmoc-Gly-OH, Fmoc-Glu (OtBu)-OH, Fmoc-Ala-OH and Boc-His(trt)-OH.

Cleavage and deprotection is one of the most crucial potential problems steps in peptide synthesis. The treatment of a peptidyl resin with a cleavage cocktail is not one simple reaction, but a series of competing reactions. Unless suitable reagents and reaction conditions are selected, the peptide can be irreversibly modified or damaged. The goal of cleavage/deprotection is to separate the peptide from the support while removing the protecting groups from the side-chains. This should be done as quickly as possible to minimize the exposure of the peptide to the cleavage reagent. The peptide is then recovered from the reaction mixture and analyzed.

Fmoc group de-protection was carried out by using 20% piperidine in DMF. Usually require 7 to 10 volumes to the batch size. As peptide synthesis grows resin volume get increased due to addition of amino acid on resin. So, to maintain proper swelling, piperidine DMF mixture need to increase gradually. Usually for complete removal of Fmoc group, it is recommended to perform two cycle of piperidine DMF mixture (10 min and 30 min) requires without washing. After piperidine-DMF treatment resin needs to wash thoroughly with DMF followed by DCM, which is further followed by DMF for a minimum five minutes to ten minutes. If required, it is suggested to repeat the process of deprotection of Fmoc and resin wash depends on the traces of Fmoc and resin. After washing check Ninhydrin test to assure complete removal of Fmoc group.

The process employed in the present invention involves the use of Wang resin, which is employed as the resin solid phase support, and said Fmoc-Gly-resin with substitution degree in the range from 0.40 to 0.75 mmol/g.

The prior art processes involves the use of amino acids in large excess as per the prior art disclosed processes it involves the molar ratio as 1:3:3:3:3 and 1:5:5:5:5, it clear indicates that which results into higher production cost, whereas the present invention involves the use of two molar equivalent except amino acid coupling in position at 15, 16, 18, 22, 24, 25 28, and 31, wherein 2 to 3 molar equivalents are required for this coupling.

The obtained Liraglutide undergo final purification and lyophilization; wherein the purification is performed by a reverse-phase high performance liquid chromatography using a reverse-phase C8 or C18 column using ammonium salts selected from selected from ammonium acetate, ammonium chloride, ammonia, ammonium bicarbonate, ammonium carbonate or combination thereof; and ammonium buffers in first purification and followed by 0.1% TFA in water, acetonitrile or mixture thereof.

The prior art processes involve the fragment condensation into each of solid phase segment needed excess fragments to condensation, which results in the serious waste of peptide fragments, resulting in the high cost of synthesis. The same solid phase segment condensation needed synthesis in multiple reactions and it condensation having limitation in resin substitution, and it generate large amount of waste. Also, the solid phase fragment condensation method of synthesis generates impurities of fragments during the condensation due to unreacted fragments. To overcome these problems the present inventors developed a process for the synthesis of Liraglutide, which is industrially feasible and free of process related impurities.

In another embodiment of the invention relates to an improved process for solid phase synthesis of highly pure Liraglutide, comprises treating crude liraglutide is finally obtained by purification and lyophilizing; wherein the purification is performed by a reverse-phase high performance liquid chromatography using a reverse-phase C8 or C18 column using ammonium buffer and ammonium salts and 0.1% TFA in water, acetonitrile or mixture thereof.

In the improved process of purification, the purification involves reverse-phase high performance liquid chromatography using a reverse-phase C8 or C18 column using ammonium salts selected from selected from ammonium acetate, ammonium chloride, ammonia, ammonium bicarbonate, ammonium carbonate or combination thereof; and ammonium buffers in first purification and followed by 0.1% TFA in water, acetonitrile or mixture thereof.

The process related impurities that appear in the impurity profile of the Liraglutide may be substantially removed by the process of the present invention resulting in the formation of substantially pure Liraglutide, which meets the ICH guidelines.

The merit of the process according to the present invention resides in that product isolated after drying is stable and having a purity of greater than or equal to 99.5% purity by HPLC, which was not disclosed in any of the prior-art. The product obtained as per the present invention is highly pure than the any of the prior-art products obtained. Still now no-publication disclosed a purity of 99.5%.

Solubility is one of the important parameters to achieve desired concentration of drug in systemic circulation for achieving required pharmacological response. Poorly soluble drugs often require high doses in order to reach therapeutic plasma concentrations after subcutaneous administration. Low solubility is the major problem encountered with formulation development of new chemical entities as well as generic formulation development. Most of the drugs are either weakly acidic or weakly basic having poor solubility. The improvement of drug solubility thereby its oral bio-availability remains one of the most challenging aspects of drug development process. The enhancement in the purity of liraglutide, which is free of process related impurities inherently, increases the solubility of liraglutide, which plays a major role for enhancement of drug activity.

The present invention also relates to a process for the preparation of liraglutide, which is substantially pure having a purity of greater or equal to 99.5% and meeting the ICH guidelines. Further, the liraglutide obtained as per the present process is found devoid of any other process related impurities and is adequately stable to handle and store for longer time (at least up to more than 6 months) without any significant or measurable change in its morphology and physicochemical characteristics.

The following examples illustrate the nature of the invention and are provided for illustrative purposes only and should not be construed to limit the scope of the invention.

### EXAMPLES

### Example-1

### Step-I: Attachment of First Amino Acid to Resin

In a round bottom flask wang resin 5 gram suspend in MDC 50 ml and kept for swelling for 4 to 5 hours, in another flask dissolve Fmoc-Gly-OH 1.5 to 2.5 equivalents (relative to resin) in 20 ml DMF and addition of same equivalent of Oxymapure and kept for stirring, In another separate flasks dissolve 0.1 equivalent DMAP in 20 ml DMF. Add 1 equivalent of DIC in the amino acid mixture prior to addition in resin flask. Finally, DMAP solution addition and keep the resin flask under stirring for 3 to 5 hours at room temperature to yield Fmoc-Gly-OH wang resin.

### Step-II: Capping of Unreacted Resin with Acetic Unhydride in Pyridine

The unreacted resin in Fmoc-Gly-OH wang resin has been capped with the 2 equivalents of acetic anhydride solution (4.1 ml acetic anhydride in 3.3 ml pyridine) by stirring the reaction mixture for 30 minutes at room temperature. Filter the resin in sintered glass funnel and wash 3 times with DMF, 3 times with MDC and finally 3 times with methanol, dry the resin under vacuum and checked the substitution level by UV method.

### Example-2

### Preparation of Fmoc-Lys-(Glu (N^{a}-Palmitoyl)-OtBu)-OH

### Step 1: Synthesis of Palmitoyl-OSu Activated Ester

Weigh 256.42 g of n-hexadecanoic acid (1.0 mol), add 115.40 g of HOSu (N-Hydroxy succinimide) (1.0 mol) to 1500 ml of Ethyl acetate in a round bottom flask, and add 206 g of DCC (1.0 mol) at room temperature, stir the mixture for 12 hours. The reaction solution was filtered; the mother liquid was distilled and found oily solid, dissolved the oily solid in n-hexane, filtered and dried to yield Palmitoyl-OSu activated ester.

### Step 2: Synthesis of Palmitoyl-Glu-OtBu

101.62 g of H-Glu-OtBu (0.5 mol) and Pamitoyl Osu 176.75 (0.5 mol) was dissolved in a 500 ml of DMF, to the solution add 129.25 g of diisopropylethyl amine, and the reaction was allowed for stirring 12 hours to 15 hours at room temperature. After completion of the reaction, add 7 volumes of water and the pH was adjusted to 3-3.5 using 10% dilute hydrochloric acid. A white precipitate was obtained and filtered. The resulting white precipitate was recrystallized using 60 ml ethyl acetate and the solid Palmitoyl-Glu-OtBu is dried.

### Step 3: Synthesis of Palmitoyl-Glu (OSu)-OtBu

88.33 g of Palmitoyl-Glu-OtBu (0.2 mol), 27.62 g of HOSu (N-Hydroxy succinimide) (0.2 mol) was added to a reaction flask containing 500 ml of Ethyl acetate, 49.51 g of DCC (0.2 mol). The obtained reaction mixture was stirred for 12 hours, at room temperature. The reaction solution filtered, mother liquor dried into rotary evaporator, recrystallized in n-hexane 3 times, to get Palmitoyl-Glu (OSu)-OtBu activated ester.

### Step 4: Synthesis of Fmoc-Lys-(Glu (N^{a}-Palmitoyl)-OtBu)-OH

36.74 g of Fmoc-Lys-OH (0.1 mol) and 53.87 g of Palmitoyl-Glu (OSu)-OtBu was weighed. was added in to a reaction flask containing 100 ml of DMF. To the solution, 2.5 mol of diisoropylethyl amine was added, and the reaction mixture was stirred for 12 hours to 15 hours at room temperature. To the obtained reaction mixture, add 7 volumes of water and then the pH was adjusted to 3-3.5 using HCl solution. A precipitate was obtained and filtered. The resulting precipitate was recrystallized in n-hexane. The solid was dried to yield Fmoc-Lys-(Glu (N^{a}-Palmitoyl)-OtBu)-OH.

Yield: 60%

### Example-3

### Preparation of Liraglutide

### Step I Deprotection

To the obtained material in example 1-step II are proceed for the solid phase synthesis of liraglutide: Taken 10-gram Fmoc-Gly-Wang resin in the peptide synthesis reaction vessel and added 100 ml 20% piperidine in DMF and stirred for 10 minutes and the solvent drained completely. Again added 20% piperidine in DMF and stirred it for 30 minutes and the solvent drained completely, followed by washing 2 times each with 60 ml DMF and 60 ml DCM for 5 minutes and then drain. Finally, resin was washed with 60 ml DMF for 5 minutes and then drain, the washed Gly-Wang resin is ready for further coupling.

### Step II Coupling

To the obtained Gly-Wang resin, add 8.3 gm of Fmoc-Arg(pbf)-OH and 2.7 gm of ethyl (Oxymapure) hydroxyimino cyanoacetate and dissolved in 50 ml DMF. To the reaction mixture, add 2.4 ml DIC and stir for 5 minutes, the resultant mixture was added in to the peptide reaction vessel and stirred for 2 to 4 hours at room temperature. The coupling is monitored by the kaiser test. After coupling the solution is drained, followed by washing 2 times with 60 ml DMF and 60 ml DCM for 5 minutes and drain every time after washing by DMF or DCM. Finally coupled resin-amino acid was washed with 60 ml DMF for 5 minutes and then drained the content. The washed Wang resin-Gly-Fmoc-Agr(pbf) is ready for further deprotection of Fmoc. Deprotection of Fmoc for Wang resin-Gly-Fmoc-Agr(pbf) has to performed using the same process as described in step I of this example. The efficacy of the coupling and deprotection is monitored by the kaiser Ninhydrin test, the coupling of the reaction is repeated if kaiser test positive.

### Step III Subsequent Deprotection and Coupling

Material obtained from step II is proceeded for sequential coupling of the amino acids as per the backbone of liraglutide that is Fmoc-Gly-OH (Gly), Fmoc-Arg(Pbf)-OH (Arg), Fmoc-Val-OH (Val), Fmoc-Leu-OH (Leu), Fmoc-Trp(Boc)-OH (Trp), Fmoc-Ala-OH (Ala), Fmoc-Ile-OH (Ile), Fmoc-Phe-OH (Phe), Fmoc-Glu(OtBu)-OH (Glu), Fmoc-Lys(Pal-Glu(OtBu))-OH, Fmoc-Ala-OH (Ala), Fmoc-Ala-OH (Ala), Fmoc-Gln(Trt)-OH (Gln), Fmoc-Gly-OH, Fmoc-Glu (OtBu)-OH, Fmoc-Leu-OH, Fmoc-Tyr (tBu)-OH, Fmoc-Ser(tBu)-OH, Fmoc-Ser(tBu)-OH (Ser), Fmoc-Val-OH, Fmoc-Asp (OtBu)-OH (Asp), Fmoc-Ser (tBu)-OH, Fmoc-Thr (tBu)-OH, Fmoc-Phe-OH, Fmoc-Thr (tBu)-OH, Fmoc-Gly-OH, Fmoc-Glu (OtBu)-OH, Fmoc-Ala-OH and Boc-His(trt)-OH.

### Step IV Coupling Reaction at Higher Temperature

During the course of sequential coupling of amino acids as per the backbone of liraglutide some of the amino acid not couples completely and hence additional reaction parameters are need, at the stage of coupling of amino acids particularly position at 15, 16, 18, 22, 24, 25, 28, 31 and amino acids are Fmoc-Gln(trt)-OH, Fmoc-Gly-OH, Fmoc-Glu(OtBu)-OH, Fmoc-Leu-OH, Fmoc-Tyr(tBu), OH, Fmoc-Val-OH, Fmoc-Ser(tBu), —OH, Fmoc-Thr(tBu)-OH, Fmoc-Gly-OH, Boc-His(trt)-OH require higher temperature in the range of 35 to 60° C. for completion of the reaction/coupling.

### Step V Cleavage of Peptide from Resin

The peptide-resin obtained from the synthesis processed for the cleavage of peptide from resin as: 10 gram of peptide-resin taken in round bottom flask and added 100 ml cocktail mixture consisting of TFA/TIPS/Water/Phenol (87.5%/5%/5%/2.5%) and stirred for 3 hours at room temperature. The reaction mixture was filtered by sintered disk and resin washed with 20 ml TFA. The obtained filtrate was added into chilled 1-liter diethyl ether under stirring and maintaining the temperature to 4° C. for 30 minutes and further cooled to 0-4° C. and stirred for 30 minutes. The precipitate obtained is filtered, washed with diethyl ether and dried at 25 to 30° C. to obtain a dry crude liraglutide.

### Example 4

### Purification of Crude Liraglutide

### Step I: Preparation of Crude Liraglutide Solution

Dissolve 20 g of crude Liraglutide in 200 ml of purified water and pH adjusted 9 to 10 using aqueous ammonia, the solution is filtered through 0.45-micron filter to remove un-dissolved particles.

### Step II: Purification I by Prep HPLC

The filtrate obtained from step I is injected into C-8 or C18 50 mm dia column of preparative HPLC and, the peptide was eluted using a gradient method of mobile phase (Buffer A and B). Composition of buffer as 20 mM ammonium acetate pH adjusted 8.5±0.25 with ammonia and labeled as mobile phase A and 100% Acetonitrile as mobile phase B. Operation of prep HPLC includes stabilization of column and maintain the flow rate of 50-70 ml/min with mobile phase A and B and same flow rate while complete operation, collect the fraction of Liraglutide and tested for content and purity by analytical HPLC. Liraglutide fractions having HPLC purity above 70% collected and evaporated the acetonitrile to get liraglutide in buffer only.

### Step III: Purification I by Prep HPLC

The aqueous solution obtained from step II is again injected in 50 mm dia column of preparative HPLC, the peptide was eluted using gradient method of 0.1% TFA in water as mobile phase A and Acetonitrile as mobile phase B with flow of 50-70 ml/min. collect the fraction of Liraglutide and analysis by analytical HPLC. Fractions having more than or equal to 99.5% purity is combined and acetonitrile evaporated to get liraglutide in aqueous medium. The aqueous solution obtained is again injected on the 50 mm dia column of preparative HPLC and washed with the buffer A as described in step II for 10 minutes and further washing with acetonitrile and water and collection of entire fractions and further process of lyophilization after removal of acetonitrile.

### Step IV: Lyophilization to get Pure Solid Liraglutide

The aqueous solution obtained from step III is freezed gradually −20° C. degree and then −40° C. prior to lyophilization. Lyophilization is carried out at −50 to −55° C. for 24 to 48 hours under vacuum. 2.0-gram pure solid liraglutide obtained after the lyophilization.

HPLC Purity: 99.5%

We claim:

1. An improved process for solid phase synthesis of highly pure Liraglutide, comprises:
a) reacting protected glycine with resin in the presence of an activating agent and a solvent to form Fmoc-Gly-resin and followed by capping with acetic anhydride in pyridine;
b) deprotection of Fmoc-Gly-resin, followed by solid-phase synthesis, amino acids with N-terminal Fmoc protection and side chain protection are sequentially coupled based on the sequence of peptide backbone of liraglutide, wherein purified Fmoc-Lys(Pal-Glu(OtBu)-OH is employed in place of Lysine; wherein the amino acids coupling is performed at 25-30° C. and involves 2 molar equivalent except amino acid coupling in position at 15, 16, 18, 22, 24, 25 28, and 31 coupling involves 2 to 3 molar equivalent and performed at 35-60° C. in presence of activating agent and solvent; and
c) Liraglutide is finally obtained by purification and lyophilizing; wherein the purification is performed by a reverse-phase high performance liquid chromatography using a reverse-phase C8 or C18 column using ammonium salts and ammonium buffers in first purification and followed by 0.1% TFA in water, acetonitrile or mixture thereof.

2. An improved process for solid phase synthesis of highly pure Liraglutide according to claim 1, wherein said step a) involves the use of Wang resin, which is employed as the resin solid phase support, and said Fmoc-Gly-resin with substitution degree in the range from 0.40 to 0.75 mmol/g.

3. An improved process for solid phase synthesis of highly pure Liraglutide according to claim 1, wherein steps a) and b) the activation agent for coupling of amino acid is of DIC (Diisopropylcarbodiimide), DCC (N,N′-Dicyclohexylcarbodiimide) Ethylcyano(hydroxyimino)acetate, DIPEA (Diisopropylethylamine), HCTU (O-(1H-6-Chlorobenzotriazole-1-yl)-1,1,3,3-tetramethyluronium hexafluorophosphate), HATU (1-[Bis(dimethylamino)methylene]-1H-1,2,3-triazolo[4,5-b]pyridinium3-oxide hexafluorophosphate.

4. An improved process for solid phase synthesis of highly pure Liraglutide according to claim 1, wherein step b) the unreacted or free amino group of amino acid after coupling is protected by acetic anhydride in pyridine.

5. An improved process for solid phase synthesis of highly pure Liraglutide according to claim 1, wherein said step b) deprotection of Fmoc-Gly-resin involves the use of DMF and piperidine.

6. An improved process for solid phase synthesis of highly pure Liraglutide according to claim 1, wherein said step b) for sequential coupling involves the use of amino acids Fmoc-Arg(Pbf)-OH (Arg), Fmoc-Gly-OH (Gly), Fmoc-Arg(Pbf)-OH (Arg), Fmoc-Val-OH (Val), Fmoc-Leu-OH (Leu), Fmoc-Trp(Boc)-OH (Trp), Fmoc-Ala-OH (Ala), Fmoc-Ile-OH (He), Fmoc-Phe-OH (Phe), Fmoc-Glu(OtBu)-OH (Glu), Fmoc-Lys(Pal-Glu(OtBu))-OH, Fmoc-Ala-OH (Ala), Fmoc-Ala-OH (Ala), Fmoc-Gln(Trt)-OH (Gln), Fmoc-Gly-OH, Fmoc-Glu (OtBu)-OH, Fmoc-Leu-OH, Fmoc-Tyr (tBu)-OH, Fmoc-Ser(tBu)-OH, Fmoc-Ser(tBu)-OH (Ser), Fmoc-Val-OH, Fmoc-Asp (OtBu)-OH (Asp), Fmoc-Ser (tBu)-OH, Fmoc-Thr (tBu)-OH, Fmoc-Phe-OH, Fmoc-Thr (tBu)-OH, Fmoc-Gly-OH, Fmoc-Glu (OtBu)-OH, Fmoc-Ala-OH and Boc-His(trt)-OH

7. An improved process for solid phase synthesis of highly pure Liraglutide according to claim 1, wherein said solvent is selected from the group consisting of DMF, DCM, NMP, Acetonitrile, TFA, Piperdine, Pyridine, Diethyl Ether, Diisopropyl Ether, Methyl tertiary Butyl Ether, Ethyl acetate, Dimethyl sulphoxide, Diisopropyl ethylamine, hexane, water and combination thereof.

8. An improved process for solid phase synthesis of highly pure Liraglutide, according to claim 1, wherein ammonium salts is selected from ammonium acetate, ammonium chloride, ammonia, ammonium bicarbonate, ammonium carbonate, ammonium formate or combination thereof.

9. An improved process for solid phase synthesis of highly pure Liraglutide, comprises treating crude liraglutide is finally obtained by purification and lyophilizing; wherein the purification is performed by a reverse-phase high performance liquid chromatography using a reverse-phase C8 or C18 column using ammonium buffer and ammonium salts and 0.1% TFA in water, acetonitrile or mixture thereof.

10. An improved process for solid phase synthesis of highly pure Liraglutide, according to claim 8, wherein ammonium salts is selected from ammonium acetate, ammonium chloride, ammonia, ammonium bicarbonate, ammonium carbonate or combination thereof.
